(19) United States
(12) Patent Application Publication
(10) Pub. No.: US 2019/0224202 A1
(43) Pub. Date: Jul. 25, 2019

(54) STABLE LIQUID FORMULATIONS OF PEMETREXED

(71) Applicant: LEIUTIS PHARMACEUTICALS PVT. LTD., Hyderabad (IN)

(72) Inventors: Kocherlakota CHANDRASHEKHAR, Secunderabad (IN); Banda NAGARAJU, Hyderabad (IN)

(73) Assignee: LEIUTIS PHARMACEUTICALS PVT. LTD., Hyderabad, TS (IN)

(21) Appl. No.: 15/735,034

(22) PCT Filed: Jun. 9, 2016
(86) PCT No.: PCT/IB2016/053391

§ 371 Date: Dec. 8, 2017

(30) Foreign Application Priority Data
Jun. 10, 2015 (IN) 2914/CHE/2015
Sep. 14, 2015 (IN) 4865/CHE/2015

(51) Int. Cl. A61K 31/519; A61K 47/18; A61K 47/26; A61K 9/00; A61K 9/08

(52) U.S. Cl. CPC A61K 31/519; A61K 47/183; A61K 9/08; A61K 9/0019; A61K 47/26

(57) ABSTRACT

The present invention provides a stable ready to use liquid parenteral formulations of Pemetrexed or a pharmaceutically acceptable salt thereof. Further this invention also describes process of preparing such formulations.

### BACKGROUND OF THE INVENTION

Pemetrexed belongs to the class of chemotherapy drugs called folate antimetabolites and is used in the treatment of malignant pleural mesothelioma and non-small cell lung cancer. Pemetrexed has the chemical name N-{4-[2-(2-Amino-4-oxo-4,7-dihydro-1H-pyrrolo[2,3-d]pyrimidin-5-yl)ethyl]benzoyl}-L-glutamic acid. The chemical structure is represented below:

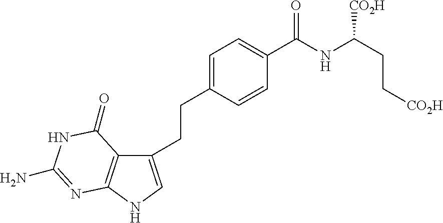 [SMILES: Nc1nc2[nH]cc(CCc3ccc(C(=O)N[C@@H](CCC(=O)O)C(=O)O)cc3)c2c(=O)[nH]1]

Pemetrexed was approved by the Food and Drug Administration (FDA) in February 2004 under the brand name Alimta® for the treatment of malignant pleural mesothelioma in combination with cisplatin. Alimta® is available as sterile lyophilized powder for intravenous infusion, in single-dose vials containing 100 mg or 500 mg equivalent Pemetrexed. In July 2004, the drug was approved by the FDA as a second line agent for the initial treatment of advanced or metastatic non-small cell lung cancer.

Pemetrexed was first disclosed in U.S. Pat. No. 5,344,932. Crystalline forms of Pemetrexed disodium are described in WO/2001/014379.

U.S. Pat. No. 7,138,521 to Chelius et al., describes a stable crystalline heptahydrate form of Pemetrexed having a characteristic X-ray diffraction pattern.

WO/2008/124485 to Raghavendracharyulu et al., discloses various crystalline forms of Pemetrexed diacid and amorphous Pemetrexed disodium.

EP 1943252 to Jonathan et al., discloses a process for the preparation of a lyophilized di-base-addition salt of Pemetrexed, in particular, a Pemetrexed disodium salt, directly from Pemetrexed diacid or an acid or base addition salt thereof.

Pemetrexed is available under the brand name Alimta® as sterile lyophilized powder for intravenous infusion, in single-dose vials. However, reconstitution of the lyophilized product is clinically inconvenient and also lyophilization process is time consuming and often incurs significant expense. Hence, there is a strong need to develop alternate formulations of Pemetrexed.

The inventors have developed ready to use liquid formulation of Pemetrexed which overcomes the disadvantages of the formulations reported in prior art.

### SUMMARY OF THE INVENTION

One aspect of the invention provides stable ready to use liquid parenteral pharmaceutical formulation comprising of Pemetrexed diacid, amino acids and water.

Yet another aspect of the invention provides stable ready to use liquid parenteral pharmaceutical formulation comprising of Pemetrexed diacid, one or more aminoacids, water and optionally other pharmaceutically acceptable adjuvants.

### DETAILED DESCRIPTION OF THE INVENTION

This invention relates to ready to use liquid parenteral pharmaceutical formulations of Pemetrexed, wherein Pemetrexed diacid is used as the active agent.

As used herein, “ready to use Pemetrexed” formulations refers to formulations that contain Pemetrexed in dissolved or solubilized form and are to be intended to be used as such or upon further dilution in intravenous diluents.

The term “about”, as used herein, refers to any value which lies within the range defined by a variation of up to ±15% of the value.

In one embodiment, ready to use liquid parenteral pharmaceutical formulations of Pemetrexed comprise:
i. Pemetrexed Diacid
ii. One or more amino acids selected from glycine, histidine, arginine, lysine or salts thereof
iii. One or more solvents.
iv. Optionally other pharmaceutically acceptable adjuvants.

In yet another embodiment, ready to use liquid parenteral pharmaceutical formulations of Pemetrexed comprise:
i. Pemetrexed Diacid
ii. One or more amino acids selected from glycine, histidine, arginine, lysine or salts thereof
iii. Water.
iv. Optionally, saccharides/sugars selected from sucrose, mannitol, glucose, fructose and chelating agents selected from DOTA, DTPA and EDTA.

In one of the preferred embodiment, ready to use liquid parenteral pharmaceutical formulations of Pemetrexed comprise:
i. Pemetrexed Diacid
ii. One or more amino acids selected from glycine, histidine, arginine, lysine or salts thereof
iii. Water wherein the percentage of amino acids in the formulation ranges from about 1% to 30% w/w, based on the total weight of the formulation.

Suitable amino acids according to the present invention include, but not limited to glycine, histidine, arginine, lysine, methionine, proline, glutamic acid or salts thereof. Percentage of amino acids in the formulation ranges from about 1% to 30% w/w, based on the total weight of the formulation. Preferably the percentage of amino acids in the formulation ranges from about 1% to 20% w/w, based on the total weight of the formulation.

The pharmaceutical compositions of the present invention may comprise one or more saccharides/sugars such as, but not limited to sucrose, glucose, xylose, galactose, fructose, lactose, maltose, mannitol, xylitol, raffinose, dextrose, trehalose and the like. Of these the preferred saccharides are sucrose and mannitol.

The pharmaceutical compositions of the present invention may comprise chelating agents such as, but not limited to DOTA (1,4,7,10-tetraazacyclododecane-1,4,7,10-tetraacetic acid), DTPA (diethylenetriaminepentaacetic acid), EDTA (Ethylenediamine tetraacetic acid), DOTRP(tetraethyl eneglycol-1,5,9-triazacyclododecane-N,N′,N″,-tris(methylenephosphonic acid), EGTA (ethylene glycol-bis(β-aminoethyl ether)-N,N,N′,N′-tetraacetic acid), HEDTA (N-(hydroxyethyl) ethylenediaminetriacetic acid) and the like.

The pharmaceutical compositions of the present invention may optionally contain one or more anti-oxidants, preservatives, buffers, pH adjusting agents, stabilizers and the like.

Pemetrexed diacid is practically insoluble in water. The inventors have surprisingly found that the presence of amino acids in the formulation yields a stable aqueous liquid formulation of Pemetrexed overcoming the disadvantages associated with prior art.

Solubility studies of Pemetrexed were performed with various amino acids alone and in combination with other excipients in water at 25° C. to check the solubility. All the below combinations were found to be clear. The data is summarized in Table 1.

TABLE 1
Solubility study of Pemetrexed diacid in various amino acids

| Quantities in mg | | | | | | | |
|---|---|---|---|---|---|---|---|
| Pemetrexed | | | | Ammonium | | Qty in ml | |
| diacid | Histidine | Arginine | Glycine | acetate | Sucrose | Water | Description |
| 100 | 100 | — | 100 | 100 | — | 2.5 | Clear |
| 100 | 100 | — | 200 | 100 | — | 2.5 | Clear |
| 100 | 100 | — | 100 | 100 | 50 | 2.5 | Clear |
| 100 | 100 | — | 200 | 100 | 50 | 2.5 | Clear |
| 100 | 100 | — | 100 | — | — | 2.5 | Clear |
| 100 | 100 | — | 100 | — | 50 | 2.5 | Clear |
| 100 | 100 | — | 50 | 100 | — | 2.5 | Clear |
| 100 | 100 | — | 50 | 100 | 50 | 2.5 | Clear |
| 50 | — | 116 | — | — | — | 1 | Clear |
| 50 | — | 133 | — | — | — | 1 | Clear |
| 50 | — | 166 | — | — | — | 1 | Clear |

Pemetrexed formulations prepared according to the invention were tested for stability under accelerated conditions. Surprisingly no significant increase in total impurities was observed even at the accelerated conditions.

TABLE 2
Stability data for the product obtained from Example 1

| | Condition | | | | | | | | | | | |
|---|---|---|---|---|---|---|---|---|---|---|---|---|
| | 2-8° C. | | | 25° C./60% RH | | | 30° C./65% RH | | | 40° C./75% RH | | |
| | 2 Wk | 1 M | 2 M | 2 Wk | 1 M | 2 M | 2 Wk | 1 M | 2 M | 2 Wk | 1 M | 2 M |
| Total Impurities | 0.31 | 0.39 | 0.40 | 0.37 | 0.34 | 0.51 | 0.35 | 0.35 | 0.56 | 0.38 | 0.44 | 0.86 |
| Assay (%) | 99.6 | 99.2 | 99.4 | 100 | 99.3 | 99.7 | 101 | 100.3 | 99.2 | 101 | 99.1 | 99.0 |
| pH | 9.38 | 9.71 | 9.78 | 9.43 | 9.7 | 9.75 | 9.50 | 9.80 | 9.74 | 9.55 | 9.76 | 9.75 |

TABLE 3
Stability data for the product obtained from Example 4

| | Condition | | | | | | | | |
|---|---|---|---|---|---|---|---|---|---|
| | | 1 M | 2 M | 1 M | 2 M | 1 M | 2 M | 1 M | 2 M |
| | Initial | 2-8° C. | | 25° C./60% RH | | 30° C./65% RH | | 40° C./75% RH | |
| pH | 6.10 | 6.15 | 6.01 | 6.13 | 6.03 | 6.12 | 6.02 | 6.09 | 6.04 |
| Osmolality | 454 | 449 | 439 | 450 | 443 | 445 | 443 | 451 | 442 |
| Total Impurities | 0.27 | 0.30 | 0.35 | 0.28 | 0.37 | 0.38 | 0.42 | 0.62 | 0.66 |

The invention further relates to a process of preparing ready to use liquid parenteral pharmaceutical formulation of Pemetrexed diacid comprising
(i) Addition of amino acid(s) and saccharide (if required), to the water.
(ii) Addition of the chelating agent (if required)
(iii) Addition of the Pemetrexed diacid to the above solution followed by stirring till a clear solution is obtained.
(iv) Addition of amino acid(s) (if required) to adjust the pH of the solution.
(v) Filtering and filling of the solution in suitable container or vials followed by stoppering and sealing of the vials.

The following examples further describe certain specific aspects and embodiments of the present invention and demonstrate the practice and advantages thereof. It is to be understood that the examples are given by way of illustration only and are not intended to limit the scope of the invention in any manner.

### Example 1

| S. No | Ingredients | Qty/vial (mg) |
|---|---|---|
| 1 | Pemetrexed diacid | 100 |
| 2 | Arginine | 200-300 |
| 3 | Sucrose | 30-80 |
| 4 | DOTA | 0.5-5 |
| 5 | Water for injection | 1.6 mL |

### Manufacturing Process:

Water for injection was taken in a compounding vessel and arginine was added and stirred, followed by the addition of sucrose. DOTA was added to the above solution and stirred. Pemetrexed Diacid was added and stirred till a clear solution was obtained. The solution was filtered, followed by stoppering and sealing of the vials.

Pemetrexed formulation prepared according to the invention was tested for stability at various conditions. The stability data of the invention formulation is summarized in Table 2. The product is tested for stability by storing at various conditions like 2-8° C., 25° C.±60% RH, 30° C.±65% RH and 40° C.±75% RH for a period of 2 months.

### Example 2

| | | | % | | % |
|---|---|---|---|---|---|
| S. No | Ingredients | Qty/Vial | w/w | Qty/Vial | w/w |
| 1 | Pemetrexed diacid | 60.00 mg | 2.2 | 60.00 mg | 2.2 |
| 2 | D-Histidine | 50.00 mg | 1.8 | 50.00 mg | 1.8 |
| 3 | Glycine | 100.00 mg | 3.7 | 100.00 mg | 3.7 |
| 4 | L-Arginine | 5.00 mg | 0.2 | 10.00 mg | 0.4 |
| 5 | L-Lysine | 5.00 mg | 0.2 | 10.00 mg | 0.4 |
| | Monohydrochloride | | | | |
| 6 | Water for Injection | 2.5 mL | 91.9 | 2.5 mL | 91.6 |

### Manufacturing Process:

Water for injection was taken in a compounding vessel and glycine and histidine were added and stirred. Pemetrexed diacid was added and stirred. L-arginine and L-lysine were added to the above solution to adjust the pH around 5.8 to 6.0. The solution was filtered, followed by stoppering and sealing of the vials.

### Example 3

| S. No | Ingredients | Qty/vial | % w/w |
|---|---|---|---|
| 1 | Pemetrexed diacid | 100 mg | 4.00 |
| 2 | Arginine | 333 mg | 13.3 |
| 3 | Sucrose | 63 mg | 2.5 |
| 4 | DOTA | 1 mg | 0.04 |
| 5 | Water for injection | 2.0 mL | 80.1 |

### Manufacturing Process:

Water for injection was taken in a compounding vessel and arginine was added and stirred, followed by the addition of sucrose. DOTA was added to the above solution and stirred. Pemetrexed Diacid was added and stirred till a clear solution was obtained. The solution was filtered, followed by stoppering and sealing of the vials.

### Example 4

| S. No | Ingredients | Qty/ml | | % w/w |
|---|---|---|---|---|
| 1 | Pemetrexed diacid | 15 | mg | 1.5 |
| 2 | L-Histidine | 12.5 | mg | 1.3 |
| 3 | Glycine | 25 | mg | 2.5 |
| 4 | L-Arginine | 2.5 | mg | 0.3 |
| 5 | L-Lysine monohydrochloride | 2.5 | mg | 0.3 |
| 6 | Water for Injection | Q.S. to 1 | mL | 100 |

### Manufacturing Process:

Water for injection was taken in a compounding vessel and glycine and histidine were added and stirred. Pemetrexed diacid was added and stirred till a clear solution was obtained. L-arginine and L-lysine were added to the above solution to adjust the pH of the solution to around 6.1. The solution was filtered, followed by stoppering and sealing of the vials. Pemetrexed formulation prepared according to the invention was tested for stability at 2-8° C., 25±2° C./60±5% RH, 30±2° C./65±5% RH and 40±2° C./75±5% RH for a period of 2 months. The data is summarized in Table 3.

1. A stable, ready to use liquid parenteral pharmaceutical formulation of Pemetrexed comprising
(i) Pemetrexed diacid
(ii) One or more amino acids
(iii) One or more solvents
(iv) Optionally other pharmaceutically acceptable adjuvants thereof.

2. The liquid parenteral pharmaceutical formulation according to claim 1, wherein the percentage of aminoacids ranges from about 1% to 30%, based on total weight of the formulation.

3. The stable, ready to use liquid parenteral pharmaceutical formulation of claim 1, wherein the amino acids are selected from the group comprising glycine, histidine, arginine, lysine, methionine, proline, glutamic acid or salts thereof.

4. The stable, ready to use liquid parenteral pharmaceutical formulation of claim 1, wherein pharmaceutically acceptable adjuvants can be selected from saccharides/sugars and/or chelating agents.

5. The stable, ready to use liquid parenteral pharmaceutical formulation of claim 4, wherein saccharides/sugars are selected from sucrose, glucose, xylose, galactose, fructose, lactose, maltose, mannitol, xylitol, raffinose, dextrose, trehalose and chelating agents are selected from DOTA, DTPA and EDTA.

6. A stable, ready to use liquid parenteral pharmaceutical formulation of Pemetrexed comprising
(i) Pemetrexed diacid
(ii) One or more amino acids selected from arginine, histidine, glycine, lysine or salts thereof.
(iii) Water and
(iv) Optionally other pharmaceutically acceptable adjuvants thereof.

7. A stable, ready to use liquid parenteral pharmaceutical formulation of Pemetrexed comprising
(i) Pemetrexed diacid
(ii) One or more amino acids selected from arginine, histidine, glycine, lysine or salts thereof.
(iii) Water
(iv) Saccharides/sugars and/or chelating agents.
